(19) United States
(12) Patent Application Publication
(10) Pub. No.: US 2014/0350480 A1
(43) Pub. Date: Nov. 27, 2014

(54) INJECTION DEVICE

(71) Applicant: Haselmeier GmbH, St. Gallen (CH)

(72) Inventor: Joachim Keitel, Esslingen (DE)

(21) Appl. No.: 14/456,895

(22) Filed: Aug. 11, 2014

Related U.S. Application Data
(63) Continuation of application No. PCT/EP2013/000358, filed on Feb. 6, 2013.

(30) Foreign Application Priority Data
Feb. 10, 2012 (DE) 20 2012 001 410.2

(51) Int. Cl. A61M 5/315

(52) U.S. Cl. CPC A61M 5/31551; A61M 5/3158; A61M 5/24
USPC 604/210

(57) ABSTRACT

An injection device defines a longitudinal center axis, a proximal direction and a distal direction. The injection device includes a housing having a receptacle for an injection fluid vessel and an operator-manipulated element configured to be rotatable to set an amount of injection fluid to be dispensed. The operator-manipulated element is further configured to move relative to the housing in the direction of the center axis in the distal direction when rotated. The operator-manipulated element further is displaceable in the proximal direction to dispense injection fluid. A feed part is connected to the operator-manipulated element in a rotatably fixed manner and is displaced in the direction of the center axis in the distal direction during the setting of the amount of injection fluid to be dispensed. The feed part is connected to the housing in a rotatably fixed manner and is displaced in the direction of the center axis in the distal direction when the injection fluid is dispensed. A blocking contour extends around the center axis in a spiral-like manner and coacts with the feed part and to block a movement of the feed part in the direction of the center axis in the proximal direction. The blocking contour has at least at one location an interruption configured to allow a movement of the feed part in the direction of the center axis in the proximal direction.

### CROSS REFERENCE TO RELATED APPLICATIONS

This application is a continuation application of international patent application PCT/EP2013/000358, filed Feb. 6, 2013, designating the United States and claiming priority from German application 20 2012 001 410.2, filed Feb. 10, 2012, and the entire content of both applications is incorporated herein by reference.

### BACKGROUND OF THE INVENTION

U.S. Pat. No. 8,747,367 makes known an injection device where a turning movement of the control element is converted via two threaded connections into an axial movement of a feed part in the distal direction. To eject the injection fluid from the vessel, the feed part is moved in the axial direction and thus acts on a dosing piston which ejects the injection fluid from the vessel.

In the case of the injection device shown in U.S. Pat. No. 8,747,367, very different quantities of injection fluid can be set. The possible quantities to be set are predetermined via a latching device which is configured such that the control element is only able to be set in positions that correspond to admissible set quantities of injection fluid. The control element jumps from positions of the operator-controlled element which correspond to inadmissible quantities of injection fluid into the nearest admissible position.

So that the operator-controlled knob jumps automatically and reliably from an intermediate position into a latching position, the latching mechanism has to be sufficiently strong and the radial latching positions have to lie sufficiently close together. However, the strength of the latching mechanism influences the torque that the user has to apply in order to turn the operator-controlled knob and set the dose. The structurally possible spacing between the latching positions is extensively predefined and is only able to be adapted to the application within very narrow limits.

### SUMMARY OF THE INVENTION

It is an object of the invention to provide an injection device wherein only predefined quantities of injection fluid are able to be ejected from the vessel and which enables good adaptation to the desired application.

The injection device of the invention defines a longitudinal center axis, a proximal direction and a distal direction. The injection device includes: a housing having a receptacle for an injection fluid vessel configured to have an injection fluid therein; an operator-manipulated element configured to be rotatable so as to set an amount of the injection fluid to be dispensed; the operator-manipulated element being further configured to move relative to the housing in the direction of the longitudinal center axis in the distal direction when being rotated; the operator-manipulated element further being configured to be displaced in the proximal direction in order to dispense injection fluid from the injection fluid vessel; a feed part configured to be connected to the operator-manipulated element in a rotatably fixed manner and to be displaced in the direction of the longitudinal center axis in the distal direction during the setting of the amount of injection fluid to be dispensed; the feed part being further configured to be connected to the housing in a rotatably fixed manner and to be displaced in the direction of the longitudinal center axis in the distal direction when the injection fluid is dispensed from the vessel; a blocking contour running around the longitudinal center axis in a spiral-like manner and configured to coact with the feed part and to block a movement of the feed part in the direction of the longitudinal center axis in the proximal direction; and, the blocking contour having at least at one location an interruption configured to allow a movement of the feed part in the direction of the longitudinal center axis in the proximal direction.

The injection device has a feed part which, when setting the quantity of injection fluid to be ejected, is moved in the distal direction and at the same time is turned about the longitudinal center axis. When the injection fluid is ejected, the feed part is moved in the direction of the longitudinal center axis in the proximal direction, but is not turned in relation to the housing. A blocking contour, which interacts with the feed part, is provided in order to ensure that only structurally predefined, allowable quantities of injection fluid are able to be ejected. The blocking contour ensures that the feed part is only able to be moved in structurally predefined positions in the direction of the longitudinal center axis without at the same time turning in relation to the housing. The blocking contour, in this case, extends in a spiral shape about the longitudinal center axis of the injection device. The development of the blocking contour, in this case, corresponds to the movement of the feed part such that the feed part abuts against the blocking contour during its helical movement in the distal direction which the feed part carries out when setting the quantity of injection fluid to be ejected. It is possible to eject injection fluid through the gap in the blocking contour. The injection fluid, in this case, can only be delivered in quantity units which are associated with the position of the gap in the blocking contour. In this case, only one single gap in the blocking contour can be provided. However, it is also possible to provide several gaps in the blocking contour which are advantageously at a constant angular spacing with respect to one another about the longitudinal center axis. Thus, for example, two gaps can be provided in the blocking contour which are offset with respect to one another by 180° about the longitudinal center axis. Four gaps which are spaced apart by 90° with respect to one another in each case can also be advantageous. The number of gaps is dependent on the application. The blocking contour extends in an advantageous manner from the position in which the feed part is located prior to setting the dose up to the position in which the feed part is located when the maximum possible dose is set. As a result of the blocking contour being independent of the latching positions of the control element, a desired latching power and a desired spacing between notches can be provided independently of the quantity units of injection fluid which can be ejected. The operating characteristics of the injection device are able to be set structurally as a result largely independently of the injectable fluid quantities.

The “proximal direction”, in this case, refers to the injection direction, that is in the direction toward a receptacle for the injection needle or the direction in which the injection fluid is ejected from the vessel. The “distal direction” refers to the opposite direction, that is away from the injection needle. The distal end of the injection device is the end which lies remote from the injection needle. The term “proximal” refers to the side of the injection device which lies facing the puncture site during an injection and the term “distal” refers to the side which lies remote from the puncture site.

When setting the quantity of injection fluid to be ejected, in an advantageous manner the control element is adjustable into at least one injection position in which an admissible quantity of injection fluid is set, and into at least one blocking position in which an inadmissible quantity of injection fluid is set. In the blocking position, the feed part advantageously abuts against the blocking contour by way of a blocking element. The blocking element and the blocking contour overlap in the axial direction and thus prevent a movement of the feed part in the direction of the longitudinal center axis in the proximal direction. In the injection position, the blocking element is advantageously arranged on the distal side of the gap in the blocking contour. The gap is realized such that it does not overlap with the blocking element in the axial direction of the injection device such that the movement of the feed part is not obstructed in the region of the gap by the blocking element in the proximal direction.

In an advantageous manner, the injection device has a latching device which latches in the injection position and which acts between the feed part and the housing. As a result, it becomes clear to the operator when the admissible injection position has been reached. A simple configuration is achieved as a result of realizing the latching device between the feed part and the housing. As a result, the latching device is only active when the quantity of injection fluid to be ejected is being set, but not, however, during the injection operation. As a result, the force required for the injection operation can be kept low. However, it can be advantageous to provide a latching device which acts during the injection operation. In an advantageous manner, the feed part has at least one latching arm which abuts against the blocking contour and forms the blocking element. The injection device, in an advantageous manner, has at least one latching elevation which is arranged adjacent the gap in the blocking contour in the circumferential direction and which forms the latching device with the latching arm. In order to ensure that the feed part is not able to be turned in relation to the housing during the injection operation, it is provided that the injection device has a longitudinal guide which connects the feed part non-rotatably to the housing during its movement in the proximal direction. A simple embodiment is produced when the injection device has at least one longitudinal web which extends parallel to the longitudinal center axis of the injection device and which forms the latching elevation and the longitudinal guide for the latching arm. In an advantageous manner, longitudinal guides are arranged on both sides of the gap in the circumferential direction such that the injection position is defined as the latching position in both turning directions.

When setting the quantity of injection fluid to be ejected, the turning movement of the feed part, which is produced as a result of the non-rotatable connection to the control element which is to be turned by the operator, brings about, in an advantageous manner, the axial movement of the feed part via a first threaded connection. The feed part, in this case, is displaced by a first travel in the distal direction. The first threaded connection includes, in an advantageous manner, an internal thread of the feed part which interacts with an external thread of a dosing piston which is connected non-rotatably to the housing. As both the feed part and the dosing piston are held non-rotatably in the housing when the injection fluid is ejected from the vessel, the first threaded connection, when the injection fluid is ejected, provides a fixed coupling between the feed part and the dosing piston, via which the axial movement of the feed part is transmitted in the proximal direction to the dosing piston.

The distal movement of the control element when setting the quantity of injection fluid to be ejected is advantageously brought about by a second threaded connection.

In an advantageous manner, the control element is realized in multiple parts and has an actuating knob and an adjustment sleeve. In an advantageous manner, the actuating knob is connected to the feed part via an entrainer. The adjustment sleeve is in particular fixedly connected to the dosing member. The actuating knob can be advantageously displaced in relation to the adjustment sleeve between a first distal position and a second proximal position. The actuating knob is connected in particular to the adjustment sleeve via a coupling, the coupling in the first distal position of the actuating knob producing a non-rotatable connection between the entrainer and the adjustment sleeve and in the second proximal position of the actuating knob allows the adjustment sleeve to rotate in relation to the entrainer. The actuating knob is sprung in particular in the direction of its distal position such that the control knob is in its distal position when the operator does not press the control knob in the proximal direction. When setting the quantity of injection fluid to be ejected, the control knob is situated in the first distal position. The adjustment sleeve and the entrainer are turned together by the operator in the position of the actuating knob. The dosing member and the feed part are also turned at the same time. In the second proximal position of the actuating knob, the adjustment sleeve can be turned in relation to the entrainer. The position of the actuating knob is achieved when the operator presses the actuating knob in the proximal direction in order to carry out an injection.

In an advantageous manner, the dosing member is connected to the housing via the second threaded connection. The turning movement of the dosing member in particular causes the dosing member and the control element to move in the distal direction by a second travel. When setting the quantity of injection fluid to be ejected, the control element is moved accordingly in the distal direction. The dosing member is moved along a helical line in the distal direction out of the housing. The second travel, in this case, is advantageously larger than the first travel. As a result of the travel for the control element being clearly greater than the travel of the feed part, only a reduced force is required to eject the injection fluid. At the same time, on account of the long path of the control element it is simple for the operator to recognize whether or not the injection fluid has already been ejected.

In an advantageous manner, the injection device has a slide which carries a thread of a third threaded connection. In an advantageous manner, the slide is connected non-rotatably to the dosing member and via the third threaded connection to the housing. When the dosing member and consequently the slide are turned in relation to the housing, the third threaded connection causes the slide to move in the distal direction of the longitudinal center axis by a third travel. The third travel, in this case, is advantageously just as long as the first travel. As an alternative, however, it can also be provided that the slide is connected non-rotatably to the housing and via the third threaded connection to the dosing member. In this case too, when the dosing member is turned in relation to the housing, the third threaded connection causes the slide then to move in a certainly purely axial manner in the distal direction of the longitudinal axis by a third travel. Here too, the third travel is advantageously precisely as long as the first travel. The third threaded connection has a gradient which, when the third threaded connection is arranged between the slide and the housing, corresponds to the gradient of the first threaded connection. If the third threaded connection is realized between the slide and the entrainer, the gradient of the third threaded connection corresponds to the difference between the first and second threaded connection such that the same travel is produced for the slide as for the feed part. In an advantageous manner the slide has an entrainer shoulder which interacts with an entrainer shoulder of the feed part and which transmits an axial movement of the slide in the proximal direction to the feed part. The feed movement for ejecting the quantity of injection fluid to be ejected is consequently effected via the slide and the third threaded connection or the first threaded connection and the third threaded connection.

### BRIEF DESCRIPTION OF THE DRAWINGS

The invention will now be described with reference to the drawings wherein:

FIG. 1 is a side view of an injection device;

FIG. 2 shows a section along the line II-II in FIG. 1;

FIG. 3 shows the injection device from FIG. 1 after setting the quantity of injection fluid to be ejected;

FIG. 4 shows a section along the line IV-IV in FIG. 3;

FIG. 5 shows an enlarged representation of the distal housing part of the injection device from FIG. 2;

FIG. 6 shows a sectional representation of a detail of the injection device from FIG. 4 in the region of the control element after pressing the actuating knob;

FIG. 7 shows a side view of the entrainer of the injection device;

FIG. 8 shows a section along the line VIII-VIII in FIG. 7;

FIG. 9 shows a section along the line IX-IX of FIG. 7;

FIG. 10 shows a side view of the dosing member of the injection device;

FIG. 11 shows a section along the line XI-XI in FIG. 10;

FIG. 12 shows a side view of the dosing member in the direction of the arrow XII in FIG. 10;

FIG. 13 shows a side view of an inner tube of the injection device;

FIG. 14 shows a section along the line XIV-XIV in FIG. 13;

FIG. 15 shows a section along the line XV-XV in FIG. 13;

FIG. 16 shows an enlarged representation of the detail XVI in FIG. 14;

FIG. 17 shows a side view of the slide of the injection device;

FIG. 18 shows a section along the line XVIII-XVIII in FIG. 17;

FIG. 19 shows a section along the line XIX-XIX in FIG. 17;

FIG. 20 shows a side view of the feed part of the injection device;

FIG. 21 shows a section along the line XXI-XXI in FIG. 20;

FIG. 22 shows a section along the line XXII-XXII in FIG. 20;

FIG. 23 shows a side view of the feed part in the direction of the arrow XXIII in FIG. 20;

FIG. 24 shows a side view of the piston rod of the dosing piston of the injection device;

FIG. 25 shows a side view in the direction of the arrow XXV in FIG. 24;

FIG. 26 shows a section through the inner tube at the level of the line XV-XV in FIG. 13 with the feed part arranged therein in the blocking position of the control element; and,

FIG. 27 shows a sectional representation corresponding to FIG. 26 with the feed part in the injection position of the control element.

### DESCRIPTION OF THE PREFERRED EMBODIMENTS OF THE INVENTION

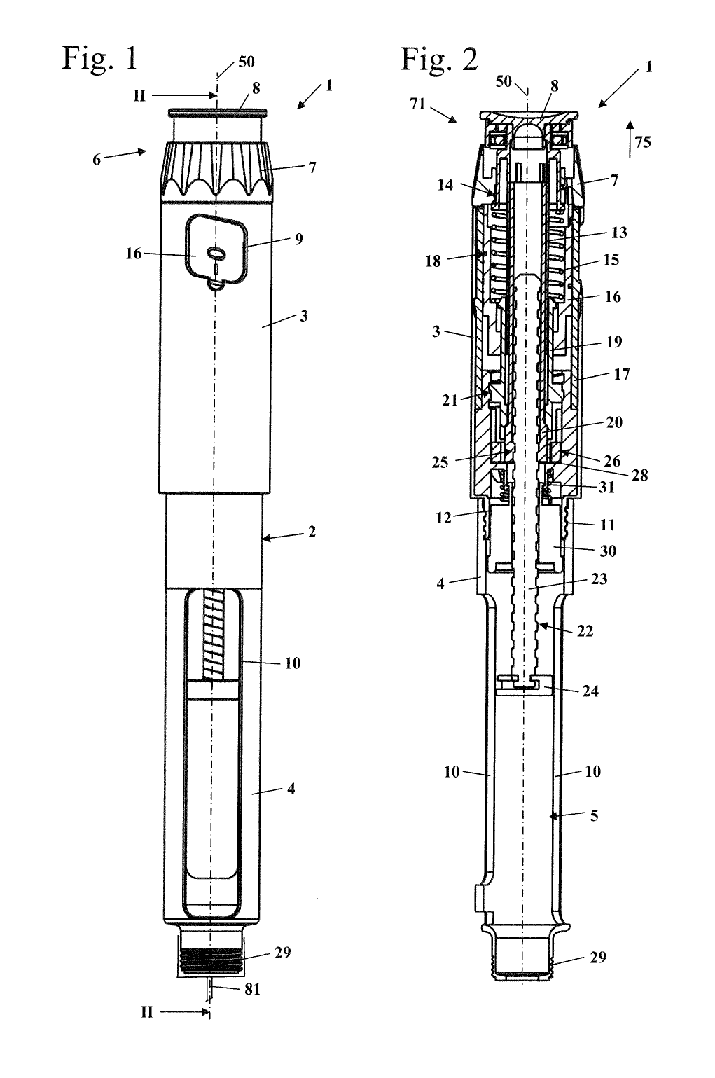

The injection device 1 shown in FIG. 1 has a housing 2 which includes an upper, distal housing part 3 and a holder 4 which is arranged on the proximal side of the upper housing part 3. At its proximal end, the holder 4 has an external thread 29, to which an injection needle 81, which is shown schematically in FIG. 1, can be screw-connected. A receptacle 5, which is shown in FIG. 2, for a vessel with injection fluid is realized in the holder 4. The vessel with injection fluid is not shown in the figures. As shown in FIG. 1, the holder 4 includes at least one recess 10, through which the vessel with injection fluid can be seen. As a result, the operator can easily recognize whether any injection fluid is still present in the vessel. As shown in FIG. 2, two recesses 10, which are arranged opposite one another, are provided on the holder 4.

As shown in FIG. 1, an operator-manipulated element 6, which has an adjustment sleeve 7 and an actuating knob 8 which is arranged on the distal end of the adjustment sleeve 7, is arranged on the distal end of the housing part 3. Adjacent the adjustment sleeve 7, the housing part 3 includes an inspection window 9 through which a scale, which is applied on a dosing member 16, can be seen. The dosing member 16 is arranged in the housing part 3. In FIG. 1 the scale shows a “0” which indicates to the user that no quantity has been set.

FIG. 2 shows the configuration of the injection device 1 in detail. The injection device 1 has a entrainer 13 which is realized in a substantially sleeve-shaped manner and which is connected axially in a fixed manner to the actuating knob 8 of the operator-manipulated element 6. In this case the term “axially” refers in each case to the direction of a longitudinal center axis 50 of the injection device 1. The actuating knob 8 is connected to the entrainer 13 via a snap-type connection which allows the actuating knob 8 to be rotated in relation to the entrainer 13. The entrainer 13 is connected to the adjustment sleeve 7 of the operator-manipulated element 6 via a coupling 14. In the case of the distal position 71 of the actuating knob 8 which is shown in FIG. 2, the coupling 14 is closed. The adjustment sleeve 7 of the operator-manipulated element 6 is connected non-rotatably to the entrainer 13. The adjustment sleeve 7 is connected fixedly to a dosing member 16 which is also designated as an adjustment member or scale tube. The entrainer 13 is connected non-rotatably to a feed part 20 which is connected to a piston rod 23 of a dosing piston 22 via a first threaded connection 25. At its proximal end the piston rod 23 carries a piston disc 24 which serves to abut against a plug of the vessel with injection fluid and via which the injection fluid is ejected from the vessel.

The piston rod 23 is held non-rotatably in a piston rod ring 30. The piston rod ring 30 is arranged so as to be axially displaceable in the injection device 1. In the position shown in FIG. 2, when no vessel is inserted into the receptacle 5, the piston rod ring 30 is pressed into its proximal position by a compression spring 31. In the position, the piston rod ring 30 can be rotated in relation to the housing part 3. If a vessel is inserted into the receptacle 5 and the holder 4 is connected to the housing part 3 at a fastening thread 11, the vessel presses the piston rod ring 30 in the distal direction. The injection device 1 has an inner tube 17 which is connected non-rotatably to the housing part 13. At its distal end, the piston rod ring 30 has a contour 12 which matches a contour of the inner tube 17. In its distal position, the piston rod ring 30 is connected non-rotatably to the inner tube 17 via the named contours and consequently also non-rotatably to the housing part 3. With the vessel inserted into the receptacle 5, the piston rod 23, as a result, is held non-rotatably in the housing part 3. As a result of the non-rotatable connection between the piston rod 23 and the housing part 3, a rotation of the feed part 20 causes the feed part 20 to move in the distal direction, that is in the direction of the arrow 75 in FIG. 2. A latching device 26, which defines latching positions of the feed part 20, is formed between the feed part 20 and the inner tube 17. In the case of the position of the feed part 20 which is shown in FIG. 2, the feed part 20 abuts against a stop 28 which is formed on the inner tube 17 and defines the position of the feed part 20 in the axial direction.

The dosing member 16 is connected to the inner tube 17 via a second threaded connection 18. The inner tube 17 is connected fixedly to the housing part 3. The inner tube could also be realized integrally with the housing part 3, however the production of the injection device 1 becomes very expensive as a result. The dosing member 16 is connected non-rotatably and axially displaceably to a slide 19 which projects into the interior of the dosing member 16. The slide 19 is connected to the inner tube 17 via a third threaded connection 21. A compression spring 15, which presses the actuating knob 8 into its first position 71, acts between the entrainer 13 and the dosing member 15.

To set the quantity of injection fluid to be ejected, the operator rotates the operator-manipulated element 6 until the desired dose appears in the inspection window 9. The adjustment sleeve 7 is turned at the same time. The dosing member 16, which is connected non-rotatably to the adjustment sleeve 7, is rotated as a result in relation to the upper housing part 3 and the inner tube 17. As a result of its rotating movement, the dosing member 16 is displaced via the second threaded connection 18 in the distal direction, that is in the direction of the arrow 75. The operator-manipulated element 6 and the entrainer 13, which is connected axially in a fixed manner to the actuating knob 8 of the operator-manipulated element 6, are moved with the dosing member 16. The operator-manipulated element 6, the entrainer 13 and the dosing member 16 are moved together in the distal direction and, at the same time, are rotated on account of the second threaded connection 18 about the longitudinal center axis 50.

The feed part 20 is also rotated in relation to the upper housing part 3 via the non-rotatable connection between the entrainer 13 and the feed part 20. The feed part 20 is also moved in the distal direction via the first threaded connection 25. The slide 19 is also moved in the distal direction as the slide 19 is connected non-rotatably to the dosing member 16. The slide 19 and the feed part 20 are also moved with a combined rotating and longitudinal movement, the path, which the slide 19 and the feed part 20 cover, being established via the first threaded connection 25 or via the third threaded connection 21. It can also be provided that the slide 19 is connected to the dosing member 16 via a third threaded connection and non-rotatably to the feed part 20.

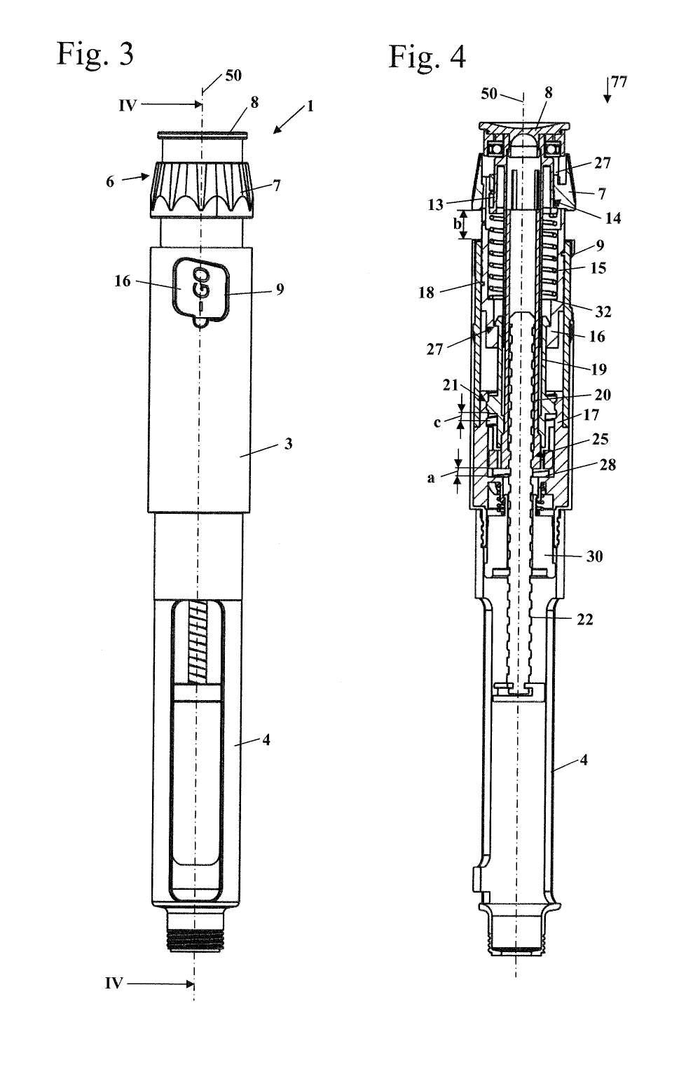

FIGS. 3 and 4 show the injection device 1 after setting the quantity of injection fluid to be ejected. The feed part 20 has moved by a first travel (a) in the distal direction. After setting the quantity of injection fluid to be ejected, the end face of the feed part 20 has moved away from the stop 28 by the first travel (a). The operator-manipulated element 6 with the adjustment sleeve 7 and the actuating knob 8 has moved in the distal direction by a second travel (b). The second travel (b) is measured in the exemplary embodiment between the proximal end face of the adjustment sleeve 7 and the distal end face of the housing part 3. The second travel (b) is clearly longer than the first travel (a). In the exemplary embodiment, the second travel (b) is a multiple, for example approximately three times the travel (a). The different travels (a) and (b) are produced by different gradients of the first threaded connection 25 and of the second threaded connection 18. The slide 19 has moved in the distal direction by a third travel (c). The third travel (c) is shown in FIG. 4 on the proximal end face of the portion of the slide 19 which carries the thread, opposite the position of the end face in FIG. 2.

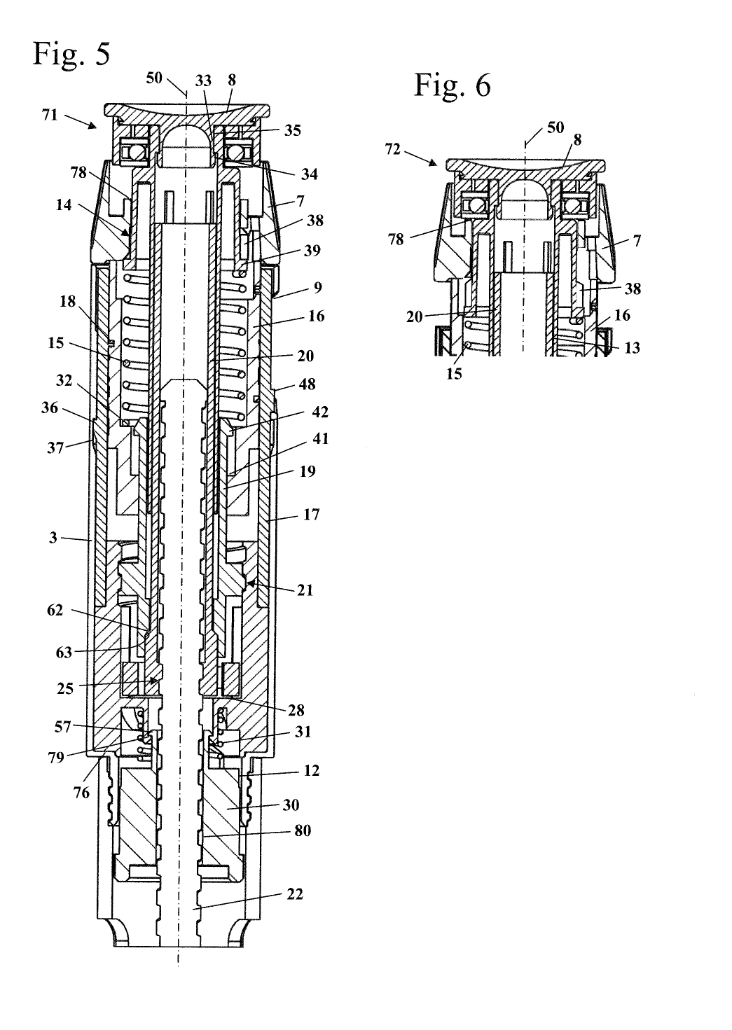

The maximum quantity of injection fluid to be set is predefined by the distance which the actuating knob 6 and the dosing member 16 can be moved in the distal direction. The distance is defined by a stop 27 (FIG. 4) which is formed between the dosing member 16 and the slide 19. As shown in FIG. 5, the dosing member 16 has at its proximal end an inwardly directed shoulder 41. The shoulder 41 is engaged behind in the axial direction by a latching edge 42 on the slide 19. The shoulder 41 forms the stop 27 with the latching edge 42. As soon as the latching edge 42 abuts against the shoulder 41, the maximum settable quantity of injection fluid is reached. The distance between the shoulder 41 and the latching edge 42 in the state of the injection device 1 shown in FIGS. 1 and 2 corresponds to the second travel (b) minus the first travel (a).

As shown in FIG. 5, the first housing part 3 has a latching indentation 37 which is realized in a circumferential manner in the exemplary embodiment. A notch 36, which is realized on the inner tube 17, projects radially outward and secures the inner tube 17 in the direction of the longitudinal center axis 50 of the injection device 1 in the housing part 3, projects into the latching indentation 37. At its proximal end, the inner tube 17 abuts against a shoulder 76 of the housing part 3. To be non-rotatably secured, the inner tube 17 has an outwardly projecting journal 48 which latches on the housing part 3 adjacent the inspection window 9.

FIG. 5 also shows the supporting of the compression spring 15. The compression spring 15 is supported by way of its proximal end on a shoulder 32 of the dosing member 16 and by way of its distal end on an edge 39 which is realized on the entrainer 13. The edge 39 projects outward from a sleeve-shaped portion of the entrainer 13. An external toothing 38 is arranged on the entrainer 13 adjacent the edge 39 on the distal side of the edge 39. The external toothing 38 interacts with an inner toothing (not shown) on the adjustment sleeve 7 and forms with the same the coupling 14. In the case of the non-actuated position of the actuating knob 8 which is shown in FIG. 5, the coupling 14 is closed and produces a non-rotatable connection between the entrainer 13 and the adjustment sleeve 7. The compression spring 15 presses the entrainer 13 in the direction of the closed position of the coupling 14. As a result, the actuating knob 8 is pressed in the direction of its distal position 71.

Once the dose to be injected has been set, an injection can be triggered. To this end, the actuating knob 8 is pressed in the direction of the arrow 77 in FIG. 4, that is in the proximal direction. As a result, the actuating knob 8 is moved against the force of the compression spring 15 in the direction of the longitudinal center axis 50 into the adjustment sleeve 7 until the actuating knob 8 abuts against a stop 78 of the adjustment sleeve 7. FIG. 6 shows the actuating knob 8 in its second, proximal position 72. In the position, the external toothing 38 of the entrainer 13 has moved out of the region of the adjustment sleeve 7. As a result, the adjustment sleeve 7 is rotatable in relation to the entrainer 13 and the actuating knob 8. The coupling 14 is open. When the actuating knob 8 is pressed further in the direction of the arrow 77 in FIG. 4, the dosing member 16 is pressed into the inner tube 17 and at the same time is displaced in the proximal direction. The dosing member 16 is rotated at the same time on account of the second threaded connection 18. As a result of the dosing member 16 rotating, the slide 19 is also rotated and is moved as a result in the proximal direction. The slide 19 has an entrainer shoulder 62 which abuts against an entrainer shoulder 63 of the feed part 20. The slide 19, during its movement in the proximal direction, presses onto the feed part 20 via the entrainer shoulders 62 and 63 and also moves the feed part in the proximal direction. The feed part 20 is connected non-rotatably to the entrainer 13 which is connected axially in a fixed manner to the non-rotating actuating knob 8. As the feed part 20 does not rotate and the dosing piston 22 is also non-rotatably connected to the housing part 3 via the piston rod ring 30, the feed part 20 and the dosing piston 22 are connected fixedly together and are moved together in the proximal direction until the feed part 20 abuts against the stop 28 and the set quantity of injection fluid has been completely ejected from the vessel.

FIGS. 7 to 25 show the components of the injection device 1 in detail. The entrainer 13 is shown in FIGS. 7 to 9. For connection to the actuating knob 8, the entrainer 13 has at its distal end inside latching elevations 35 which engage behind a latching edge 34, which is formed on a connecting piece 33 of the actuating knob 8 and is shown in FIG. 5, and as a result connect the actuating knob 8 in the axial direction to the entrainer 13. In the exemplary embodiment the sleeve-shaped entrainer 13 has on its inner periphery four guide webs 40 which extend in the axial direction. The guide webs 40 are adapted to longitudinal grooves 64 of the feed part 20 which are shown in FIG. 20 and they engage in the longitudinal grooves. With the longitudinal grooves 64, the guide webs 40 produce the non-rotatable connection between the entrainer 13 and the feed part 20. The guide webs 40 are freely displaceable in the longitudinal grooves 64 in the direction of the longitudinal center axis 50 of the injection device 1.

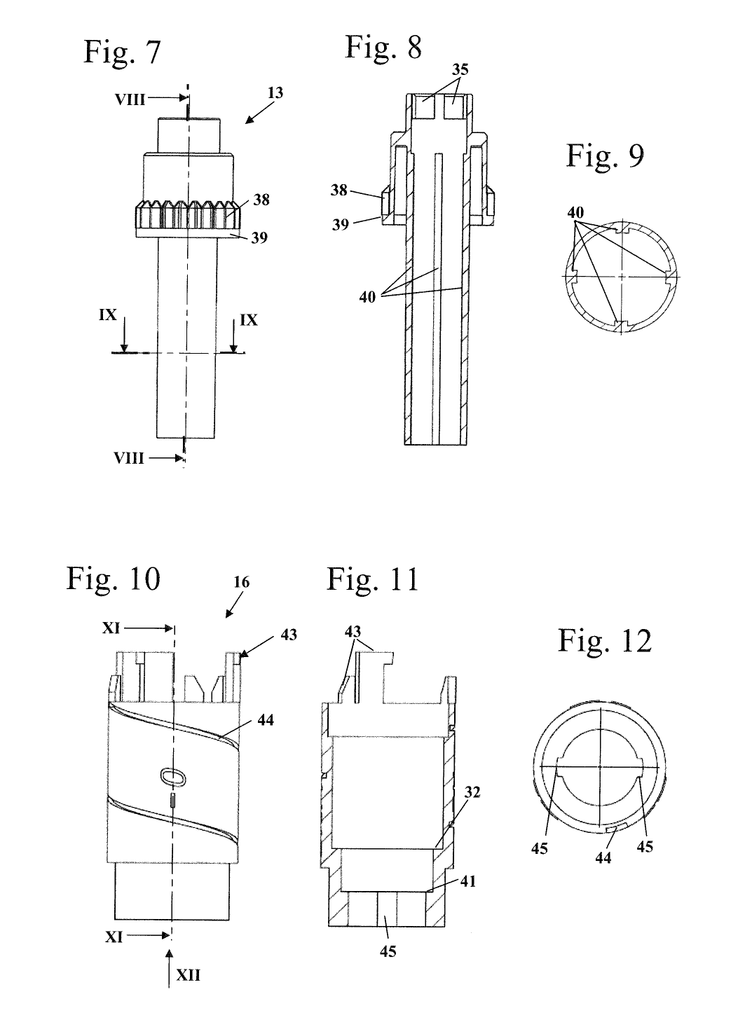

FIGS. 10 to 12 show the dosing member 16 which is also designated as the scale tube or adjustment member. The dosing member 16 is realized in a sleeve-shaped manner and has on its outer periphery an external thread 44. The external thread 44 is realized as a groove which extends in a helical manner on the outer periphery of the dosing member 16. At its distal end the dosing member 16 carries a connecting contour 43 which is formed from hook-shaped and ramp-shaped elements which produce a non-rotatable connection to the adjustment sleeve 7. As is shown in FIGS. 11 and 12, the dosing member 16 has on its proximal end two guide grooves 45 which extend parallel to the longitudinal center axis 50. The guide grooves 45 are arranged located opposite one another and interact with longitudinal webs 59 of the slide 19 which are shown in FIGS. 17 and 19. A non-rotatable connection between the dosing member 16 and the slide 19 is produced via the longitudinal webs 59 which are guided in the guide grooves 45. The longitudinal webs 59 are movable freely in the guide grooves 45 in the direction of the longitudinal center axis 50 such that the slide 19 is displaceable in relation to the dosing member 16 in the direction of the longitudinal center axis 50.

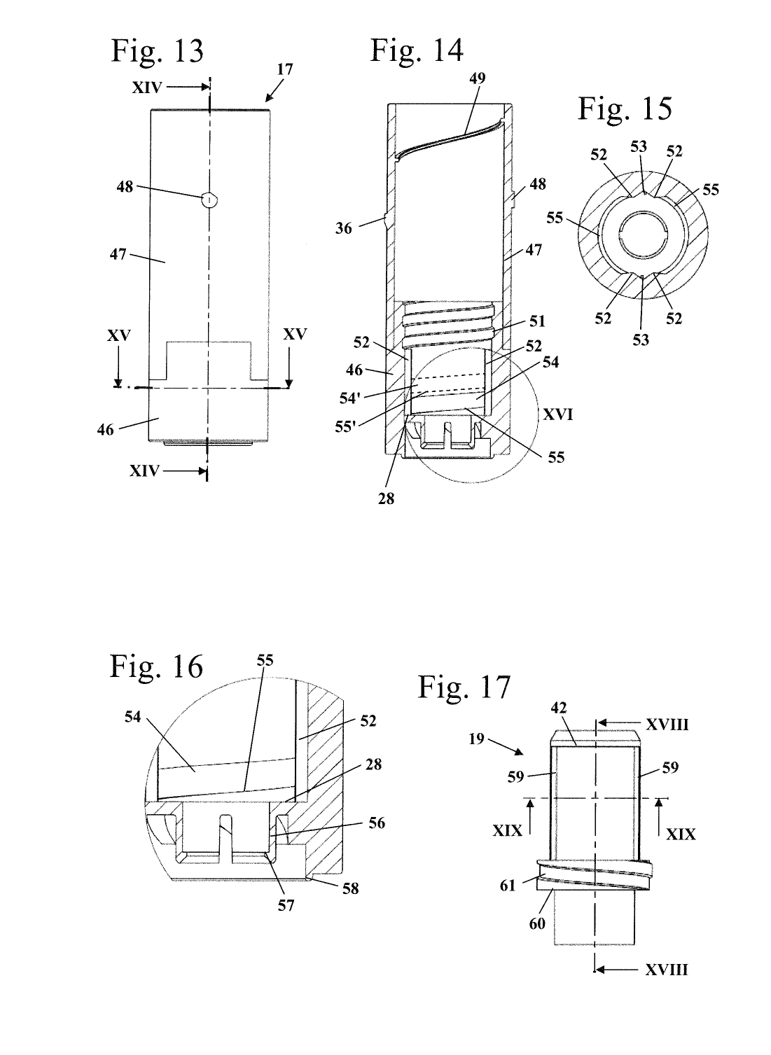

The inner tube 17 is shown in FIGS. 13 to 16. The inner tube 17 is realized in two parts and includes a proximal part 46 and a distal part 47 which are fixedly connected together. The inner tube 17 can also be produced as one part. However, the inner tube 17 is clearly more expensive to produce as a result. In order to simplify the production further, it can be advantageous to realize the inner tube 17 from more than two individual parts.

An internal thread 49, which is formed from a web which extends in a spiral manner on the inner periphery, is arranged in the distal part 47 of the inner tube 17. The internal thread 49 is formed by one single thread. It can be provided that the internal thread 49 is realized by only one or several part portions of a thread. The internal thread 49 interacts with the external thread 44 of the dosing member 16 and brings about an axial displacement of the dosing member 16 when the dosing member 16 is rotated. An internal thread 51, which interacts with an external thread 60 of the slide 19 which is shown in FIG. 17, is realized in the proximal part 46 of the inner tube 17. The third threaded connection 21 is formed by the internal thread 51 with the external thread 61. Longitudinal webs 52 connect to the proximal side of the internal thread 51. As shown in FIG. 15, a total of four longitudinal webs 52 are provided which are arranged in each case on both sides of a gap 53. Two gaps 53 which are arranged located opposite one another, that is spaced apart from one another by a circumferential angle of 180°, are provided in the exemplary embodiment.

A helical groove 54, which is realized as an indentation in the wall of the inner tube 17, is realized on the inner tube 17 at the proximal end of the interior of the inner tube 17, connecting to the stop 28 in the distal direction. A further groove 54, which is not visible in the representation, is provided in the half shell of the inner tube which lies in front of the cutting plane in FIG. 14. The helical groove 54 extends over half a thread in the exemplary embodiment. The groove 54, however, can also extend over one or several threads, as indicated schematically in FIG. 14 by the groove 54′ which is shown by a dotted line. The proximal wall of the groove 54 forms a blocking contour 55. A wall of the groove 54′ forms a blocking contour 55′ in a corresponding manner. The blocking contour 55 will be described in more detail below.

As shown in FIG. 16, a centering edge 58 projects in the proximal direction at the proximal end of the inner tube 17. The centering edge 58 projects into a proximal opening of the housing part 3 and ensures a fixed seat of the inner tube 17 in the housing part 3. Holding connecting pieces 56, on the proximal end of which radially inwardly projecting latching edges 57 are integrally formed, project additionally in the proximal direction on the proximal side of the inner tube 17. The latching edges 57 interact with a latching edge 79 of the piston rod ring 30 which is shown in FIG. 5. The latching edge 79 provides with the latching edge 57 an axial locking mechanism for the piston rod ring 30. As shown in FIG. 5, the second compression spring 31 presses the piston rod ring 30 into its proximal position until the latching edge 79 abuts against the latching edge 57. In the position, the operator is able to turn the housing part 3 in relation to the piston rod ring 30 in order to move the dosing piston 22 in the distal direction. This is provided for changing a vessel for injection fluid.

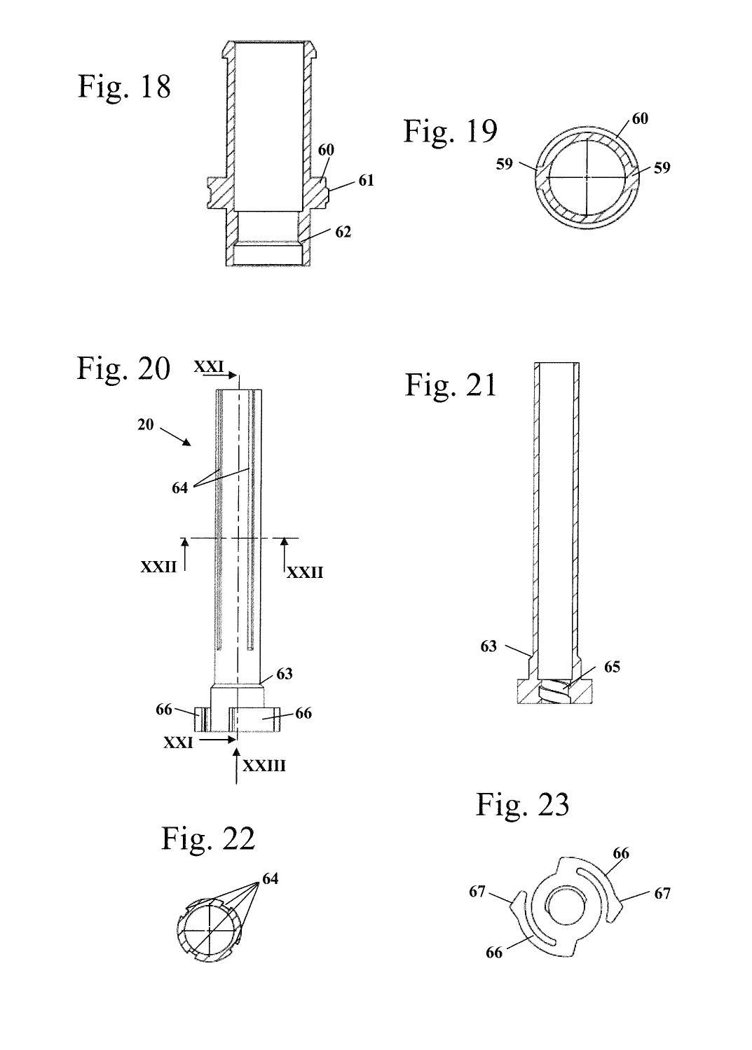

The slide 19 is shown in detail in FIGS. 17 to 19. The slide 19 has the latching edge 42 at its distal end. As shown in FIGS. 17 and 18, the external thread 61 is realized in an annular web 60 which projects radially outward. The slide 19 is also realized in a substantially sleeve-shaped manner.

The feed part 20 is shown in FIGS. 20 to 23. At its proximal end, the feed part 20 has two latching arms 66 which are shown in FIG. 23. On their free end the latching arms 66 have in each case a notch 67 which points radially outward. The latching arms 66 extend approximately in the circumferential direction and are realized so as to be sprung in a radially outward manner. FIG. 21 shows an internal thread 65 which is realized at the proximal end of the feed part 30 and interacts with the dosing piston 22. The internal thread 65 and the latching arms 66 are arranged in the same longitudinal portion of the feed part 20.

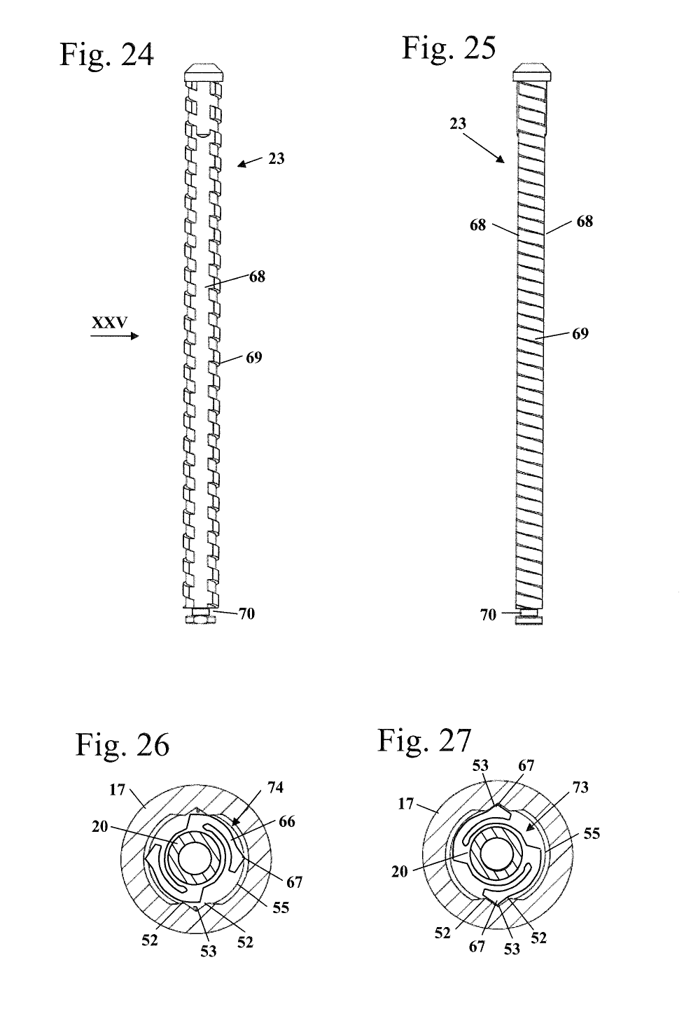

As shown in FIGS. 24 and 25, the piston rod 23 has an external thread 69 which interacts with the internal thread 65 of the feed part 20 and with the same forms the first threaded connection 25. On its opposite longitudinal sides the piston rod 23 has flattenings 68 which interact with corresponding flattenings of an opening 80 shown in FIG. 5 in the piston rod ring 30 for securing the turning position of the piston rod 23. On its proximal end the piston rod 23 has a fastening groove 70 on which the piston disc 24 is held.

FIG. 26 shows the arrangement of the feed part 20 on the blocking contour 55 with the operator-manipulated element 6 and the feed part 20 in a blocking position 74. In the position, the notches 67 of the latching arms 66 abut against the blocking contour 55. As the notches 67 and the blocking contour 55 overlap when viewed in the direction of the longitudinal center axis 50, the feed part 20 cannot be displaced in the proximal direction. The blocking position 74 of the operator-manipulated element 6 is provided when an inadmissible quantity of injection fluid is set. The ejection of an inadmissible quantity of injection fluid is prevented in a structural manner by the blocking contour 55.

If the operator-manipulated element 6, and consequently also the feed part 20, is turned further, the notches 67, after overcoming the longitudinal webs 52 which form the latching device 26 with the notches 67, move into the region of the gaps 53. The blocking contour 55 is interrupted at the gaps 53. The gaps 53 are realized as longitudinal grooves which extend parallel to the longitudinal center axis 50. Longitudinal webs 52 are arranged in the circumferential direction on both sides of the gaps 53 such that a defined latching position is produced for the feed part 20 in the injection position 73 which is shown in FIG. 27. The feed part 20 is able to be pressed in the proximal direction in the injection position 73. At the same time, the notches 67 slide in the proximal direction between the longitudinal webs 52 which extend parallel to the longitudinal center axis 50. When an injection is triggered by pressing the control knob 8, the longitudinal webs 52 prevent the feed part 20 turning during the injection. As a result, the entrainer 13 is also held in a non-rotatable manner in the housing 2 during the injection and is not able to co-rotate with the forces which prevail during the injection.

Not until the operator-manipulated element 6, and consequently also the feed part 20, is situated in the injection position 73 can the control knob 8 be displaced in the proximal direction from its proximal position 72 together with the adjustment sleeve 7, the dosing piston 16, the slide 19 and the feed part 20. As a result, the piston rod 23 is moved in the proximal direction and injection fluid is ejected from the vessel. If the operator-manipulated element 6, and consequently the feed part 20, is situated in the blocking position 74, the actuating knob 8 is certainly able to be pressed into the adjustment sleeve 7, however further displacement of the actuating knob 8 in the proximal direction in relation to the housing part 3 is not possible, but rather is blocked on account of the blocking contour 55 which blocks axial displacement of the dosing member 16, the slide 19 and the feed part 20 via the notches 67 of the latching arms 66. As a result, it can be ensured that only a structurally pre-defined, admissible quantity of injection fluid is able to be ejected from the vessel.

An injection device 1 where only one single pre-defined quantity of injection fluid is able to be ejected from the vessel is shown in the exemplary embodiment. The quantity is achieved when the control element has been turned by 180°. However, it can also be provided that several injection positions 73 which are associated with different quantities of injection fluid are possible. In this connection, the gaps 53 are to be provided at the positions on the blocking contour 55 which are associated with the desired, admissible quantities of injection fluid.

It is understood that the foregoing description is that of the preferred embodiments of the invention and that various changes and modifications may be made thereto without departing from the spirit and scope of the invention as defined in the appended claims.

What is claimed is:

1. An injection device defining a longitudinal center axis, a proximal direction and a distal direction, the injection device comprising:
a housing having a receptacle for an injection fluid vessel configured to have an injection fluid therein;
an operator-manipulated element configured to be rotatable so as to set an amount of said injection fluid to be dispensed;
said operator-manipulated element being further configured to move relative to said housing in the direction of said longitudinal center axis in said distal direction when being rotated;
said operator-manipulated element further being configured to be displaced in said proximal direction in order to dispense injection fluid from said injection fluid vessel;
a feed part configured to be connected to said operator-manipulated element in a rotatably fixed manner and to be displaced in the direction of said longitudinal center axis in said distal direction during the setting of the amount of injection fluid to be dispensed;
said feed part being further configured to be connected to said housing in a rotatably fixed manner and to be displaced in the direction of said longitudinal center axis in said distal direction when said injection fluid is dispensed from said vessel;
a blocking contour running around said longitudinal center axis in a spiral-like manner and configured to coact with said feed part and to block a movement of said feed part in the direction of said longitudinal center axis in said proximal direction; and,
said blocking contour having at least at one location an interruption configured to allow a movement of the feed part in the direction of the longitudinal center axis in the proximal direction.

2. The injection device of claim 1, wherein:
said operator-manipulated element is configured, when setting said amount of injection fluid to be dispensed, to be set to at least one injection position whereat a permissible amount of injection fluid is set and is further configured to be set to at least one blocking position whereat an impermissible amount of injection fluid is set; and,
said feed part is configured to bear against said blocking contour in said blocking position and to be disposed on the distal side of said interruption of said blocking contour in said injection position.

3. The injection device of claim 2 further comprising a latching unit configured to latch in said injection position and to act between said feed part and said housing.

4. The injection device of claim 3 further comprising:
at least one latch projection arranged adjacent to said interruption of said blocking contour;
said feed part having at least one latch arm configured to lie against said blocking contour; and,
said latch projection and said latch arm conjointly defining said latching unit.

5. The injection device of claim 4 further comprising a longitudinal guide configured to connect said feed part to said housing in a rotatably fixed manner during a movement of said feed part in said proximal direction.

6. The injection device of claim 5 further comprising a longitudinal strut running parallel to said longitudinal center axis and forming said latch projection and said longitudinal guide.

7. The injection device of claim 1 further comprising:
a first threaded connection;
said feed part being configured to perform a rotating movement during the setting of said amount of injection fluid to be dispensed; and,
said feed part being further configured to move axially during said rotating movement so as to cause said feed part to be displaced in the distal direction by a first positioning distance (a).

8. The injection device of claim 7 further comprising:
a dosing piston connected to said housing in a rotational fixed manner and having an outer thread;
said first threaded connection including an inner thread of said feed part; and,
said inner thread of said feed part being configured to coact with said outer thread of said dosing piston.

9. The injection device of claim 7 further comprising:
an entrainer;
a dosing member;
said operator-manipulated element being configured as a multi-part operating element including an actuation button and an adjustment sleeve;
said actuation button being connected to said feed part via said entrainer; and,
said adjustment sleeve being fixedly connected to said dosing member.

10. The injection device of claim 9 further comprising:
a coupling;
said actuation button being connected to said adjustment sleeve via said coupling;
said actuation button defining a first distal position and a second proximal position;
said coupling being configured to produce a rotatably fixed connection between said entrainer and said adjustment sleeve; and,
said coupling being further configured to permit a rotation of said adjustment sleeve relative to said entrainer in said second proximal position.

11. The injection device of claim 9, wherein:
said dosing member is connected to said housing via a second threaded connection;
said dosing member is configured to perform a rotational movement;
said dosing member and said operator-manipulated element are configured to move in said distal direction by a second positioning distance (b) in response to said rotational movement of said dosing member; and,
said second positioning distance (b) is greater than said first positioning distance (a).

12. The injection device of claim 9 further comprising a slide configured to carry a thread of a third threaded connection and to perform a rotational movement configured to effect a movement in said distal direction of said longitudinal center axis having a third positioning distance (c) which corresponds to said first positioning distance (a).

13. The injection device of claim 12, wherein said slide is connected to said dosing member in a rotatably fixed manner and connected to said housing via said third threaded connection.

14. The injection device of claim 12, wherein:
said feed part has a first entrainer shoulder; and,
said slide has a second entrainer shoulder configured to coact with said first entrainer shoulder and to transfer an axial movement of said slide in said proximal direction to said feed part.
